(19) United States
(12) Patent Application Publication
(10) Pub. No.: US 2017/0258985 A1
(43) Pub. Date: Sep. 14, 2017

(54) SYRINGE INFUSION PUMP SECURITY

(71) Applicant: Smiths Medical ASD, Inc., Plymouth, MN (US)

(72) Inventors: Grant A. ADAMS, Anoka, MN (US); Christopher Allen LACY, Arden Hills, MN (US); Jennifer Lynn PAVLOVIC, Middleton, WI (US); Stacy Ann SCHULTZ, Roseville, MN (US)

(73) Assignee: Smiths Medical ASD, Inc., Plymouth, MN (US)

(21) Appl. No.: 15/532,018

(22) PCT Filed: Nov. 12, 2015
(86) PCT No.: PCT/US2015/060271

§ 371 Date: May 31, 2017

Related U.S. Application Data
(60) Provisional application No. 62/086,551, filed on Dec. 2, 2014.

(51) Int. Cl. A61M 5/145; A61B 50/00; A61M 5/315

(52) U.S. Cl. CPC A61M 5/1452; A61M 5/1458; A61M 5/31576; A61B 50/00; A61B 2050/007; A61B 2050/0076

(57) ABSTRACT

An infusion pump can include a syringe receptacle, a retention mechanism, a drive mechanism, and a keyed lock. The retention mechanism can inhibit removal of the syringe from the receptacle when in an engaged state, and enable removal of the syringe when in a disengaged state. The drive mechanism can include a pusher to selectively couple to a thumb press of the syringe, a motor coupleable to the pusher, and a clutch to couple and decouple the motor to the pusher. The keyed lock can operate in coordination with the retention and drive mechanisms such that, if: the keyed lock is locked, the retention mechanism is in the engaged state, and the pusher is coupled to the thumb press of the syringe, then as long as the keyed lock remains locked, the retention mechanism is constrained to the engaged state and the clutch to the coupled state.

### TECHNICAL FIELD

This disclosure relates to infusion pumps, and more particularly, to systems and methods for syringe pump security.

### BACKGROUND

Syringe infusion pumps (or, “syringe pumps”) have demonstrated their usefulness to medical practitioners. In some cases, syringe pumps are used to deliver controlled substances such as narcotic analgesics to patients. Narcotics are often kept under lock and key in efforts to prevent their misappropriation. A desire or requirement to maintain such physical control of narcotics (or any other controlled substances) may extend to drug delivery devices, such as syringe pumps. In addition to preventing theft or diversion of drugs away from an intended patient, it may also be desirable to prevent inappropriate dosing to a patient, as might occur, for example, by improper manual manipulation of a syringe in a syringe pump that is otherwise properly configured to deliver a drug to the patient.

Some known approaches to providing secure control of drugs delivered via syringe pumps involve lock-boxes that each can enclose essentially an entire pump. While this approach can be effective, it may be burdensome for medical practitioners and can require substantially more space than unenclosed pumps. In view of the goal of simplifying workloads, it would be desirable to provide improved systems and methods for providing security for syringe pumps.

### SUMMARY

This disclosure relates to infusion pumps, and more particularly, to systems and methods for syringe pump security.

In an illustrative but non-limiting example, the disclosure provides an infusion pump that can include a receptacle configured to receive a syringe, a retention mechanism, a drive mechanism, and a keyed lock. The retention mechanism can be configured to inhibit removal of the syringe from the receptacle when in an engaged state, and to enable removal of the syringe from the receptacle when in a disengaged state. The drive mechanism can include a pusher configured to selectively couple to a thumb press of the syringe, a motor operatively coupleable to the pusher, a clutch configured to couple the motor to the pusher in a coupled state, and decouple the motor from the pusher in a decoupled state. The keyed lock can be configured to operate in coordination with the retention mechanism and the drive mechanism such that, if: (a) the keyed lock is locked, (b) the retention mechanism is in the engaged state, and (c) the pusher is coupled to the thumb press of the syringe, then as long as the keyed lock remains locked, the retention mechanism is constrained to the engaged state and the clutch is constrained to the coupled state.

In some cases, the pusher can include a pusher head, with the pusher head having a pushing surface configured to bear against the thumb press in an expulsion direction, and the pusher head further having at least one thumb press retaining clip. The thumb press retaining clip(s) can be configured to retain the thumb press against the pushing surface when engaged, and not retain the thumb press against the pushing surface when disengaged. Furthermore, the thumb press retaining clip(s) can be mechanically linked to the clutch such that the thumb press retaining clip(s) is/are engaged when the clutch is in the coupled state, and is/are disengaged when the clutch is in the decoupled state.

In some cases, the keyed lock can be disposed on the pusher.

In some cases, the infusion pump can further include a clutch release actuator actuatable to move the clutch between the coupled and decoupled states. The keyed lock can be lockable to maintain the clutch release actuator in a clutch-coupled position and unlockable to enable manipulation of the clutch release actuator between clutch-coupled and clutch-uncoupled positions.

In some cases, the infusion pump can further include a linkage mechanism mechanically coupling the clutch release actuator with the retention mechanism, such that when the clutch release actuator is in a clutch-decoupled position, the retention mechanism is not constrained to the engaged state.

In some configurations, the pusher can include a pusher rod slidably mounted relative to the receptacle, a pusher head mounted at a first end of the pusher rod (the pusher head configured to exert force on the thumb press in an expulsion direction), and a retention releaser secured relative to the pusher rod. In such a configuration the pusher, when the clutch is in the decoupled state, can be retracted such that the pusher head is retracted away from the receptacle and the retention releaser can be drawn toward the retention mechanism. Further, when the pusher is retracted essentially to a fully-retracted position, the retention releaser can be brought into proximity of the retention mechanism and release the retention mechanism from the engaged state.

In some cases, the keyed lock can be lockable to maintain the retention mechanism in the engaged state, and unlockable to enable release of the retention mechanism from the engaged state. Furthermore, the pump in such cases can further include a linkage mechanism mechanically coupling the retention mechanism with the clutch such that when the retention mechanism is in the engaged state, the clutch is constrained to the coupled state, and when the retention mechanism is in the disengaged state, the clutch is not constrained to the coupled state.

In some cases, the retention mechanism of the pump can include a clamp assembly configured to releasably secure the syringe in the receptacle. The clamp assembly can include a clamp grip configured to bear against a barrel of the syringe, and when so bearing, exert a retaining force on the syringe into the receptacle; a clamp arm connected to the clamp grip, the clamp arm slidably mounted relative to the receptacle such that the clamp grip is translatable inward and outward relative to the receptacle; and a clamp lock mechanism configured to releasably maintain the clamp arm at a syringe retention position where the clamp grip exerts the retaining force on the syringe. In some of these cases, the pump can further include a receptacle flap pivotably attached to the pump, the flap pivotable between a closed mode and an open mode. When in the closed mode, the receptacle flap can be in a position that substantially inhibits access to the receptacle and to the syringe received by the receptacle, and when in the open mode, the receptacle flap can be in a position that substantially clears access to the receptacle and to the syringe received by the receptacle. Further, the receptacle flap and clamp assembly can be structured and configured such that the clamp assembly, when the clamp grip is bearing against the barrel of the syringe, constrains the receptacle flap to the closed mode. Alternately, the receptacle flap and clamp assembly can be structured and configured such that the clamp assembly, when the clamp grip is bearing against the barrel of the syringe, does not constrain the receptacle flap to the closed mode.

In some instances, the pump further includes a receptacle flap pivotably attached to the pump, the flap pivotable between a closed mode and an open mode. When in the closed mode, the receptacle flap can be in a position that substantially inhibits access to the receptacle and to the syringe received by the receptacle, and when in the open mode, the receptacle flap can be in a position that substantially clears access to the receptacle and to the syringe received by the receptacle. The pump can further include a flap latch configured to selectively constrain the receptacle flap to the closed mode, such that the receptacle flap and flap latch form at least part of the retention mechanism. The receptacle flap can be pivotably attached to the pump via a flap release mechanism configured to enable the receptacle flap to be selectively attached to and separably released from the pump. The flap release mechanism can be structured such that it is actuatable to selectively release the receptacle flap relative to the pump only when the receptacle flap is in the open mode, and is not actuatable to release the receptacle flap when the receptacle flap is in the closed mode.

In another illustrative but non-limiting example, the disclosure provides an infusion pump that can include a receptacle configured to receive a syringe, a clamp assembly configured to releasably secure the syringe in the receptacle, and a drive mechanism. The clamp assembly can include a clamp grip configured to bear against a barrel of the syringe, and when so bearing, exert a retaining force on the syringe into the receptacle; a clamp arm connected to the clamp grip, the clamp arm slidably mounted relative to the receptacle such that the clamp grip is translatable inward and outward relative to the receptacle; and a clamp lock mechanism configured to releasably maintain the clamp arm at a syringe retention position where the clamp grip exerts the retaining force on the syringe. The drive mechanism can include a pusher device, a lead screw, a motor coupled to the lead screw, and a clutch mounted to the clutch carriage and configured to couple the pusher device to the lead screw in a coupled state and decouple the pusher device from the lead screw in a decoupled state. The pusher device can include a pusher rod slidably mounted relative to the receptacle; a pusher head mounted at a first end of the pusher rod, the pusher head configured to exert force on a thumb press of the syringe; and a clutch carriage mounted at a second end of the pusher rod, the clutch carriage including a clamp lock release structure. The pusher device, when the clutch is in the decoupled state, can be retracted such that the pusher head is drawn away from the receptacle and the clutch carriage is drawn toward the clamp lock mechanism. Further, when the pusher device is retracted essentially to a fully-retracted position, the clamp lock release structure can be brought into proximity of the clamp lock mechanism and can actuate the clamp lock mechanism to release the clamp arm.

In some cases, the pump can further include a keyed lock, where the clutch can be lockable to the coupled state by the keyed lock. The keyed lock can be provided with the pusher head. In some cases, the pump can further include an actuatable clutch release actuator which can be lockable to a clutch-coupled position by the keyed lock.

In some cases, the clamp lock mechanism can include a ratchet.

In yet another illustrative but non-limiting example, the disclosure provides an infusion pump that can include a receptacle configured to receive a syringe, a clamp assembly configured to releasably secure the syringe in the receptacle, a drive mechanism, a linkage mechanism, and a keyed lock. The clamp assembly can include a clamp grip configured to bear against a barrel of the syringe, and when so bearing, exert a retaining force on the syringe into the receptacle; a clamp arm connected to the clamp grip, the clamp arm slidably mounted relative to the receptacle such that the clamp grip is translatable inward and outward relative to the receptacle; and a clamp lock mechanism configured to releasably maintain the clamp arm at a syringe retention position where the clamp grip exerts the retaining force on the syringe. The drive mechanism can include a pusher configured to selectively couple to a thumb press of the syringe; a motor operatively coupleable to the pusher; and a clutch configured to couple the motor to the pusher in a coupled state, and decouple the motor from the pusher in a decoupled state. The linkage mechanism can be configured to couple the clutch and the clamp lock mechanism such that as the clutch is moved between the coupled and decoupled states, the clamp lock mechanism is correspondingly moved between locked and released states. The clutch, clamp lock mechanism, and linkage mechanism can be configured such that the clutch and clamp lock mechanism are lockable via the keyed lock to the coupled state and locked state, respectively.

In some cases the linkage mechanism can include a shaft rotatable between a first position and a second position and a clutch-actuating cam coupled to the shaft and configured such that the first and second positions of the shaft correspond to the coupled and decoupled states of the clutch. The linkage mechanism also can include a release cable coupled to the clamp lock mechanism configured to release the clamp arm when an end of the cable coupled to the clamp lock mechanism is displaced to a release point and an arm, rotatably coupled to the shaft and coupled to the release cable such that the arm displaces the clamp release cable correspondingly with rotations of the shaft, the second position of the shaft corresponding to displacement of the cable at least to the release point, the first position of the shaft corresponding to displacement of the cable insufficient to release the clamp arm.

In some cases, the keyed lock is disposed on the pusher.

In still another illustrative but non-limiting example, the disclosure provides an infusion pump that can include a receptacle configured to receive a syringe, a retention mechanism configured to inhibit removal of the syringe from the receptacle when in an engaged state, and to enable removal of the syringe from the receptacle when in a disengaged state, a drive mechanism, and a keyed lock. The drive mechanism can include a pusher configured to selectively couple to a thumb press of the syringe, a motor operatively coupleable to the pusher, and a clutch configured to couple the motor to the pusher in a coupled state, and decouple the motor from the pusher in a decoupled state. The keyed lock can be configured to operate in coordination with the retention mechanism and the drive mechanism such that, if: (a) the keyed lock is locked, and (b) the retention mechanism is in the engaged state, then as long as the keyed lock remains locked, the retention mechanism is constrained to the engaged state and the clutch is constrained to the coupled state.

In still yet another illustrative but non-limiting example, the disclosure provides an infusion pump that can include a receptacle configured to receive a syringe, a receptacle flap pivotably attached to the pump, the flap pivotable between a closed mode and an open mode, a flap latch configured to selectively constrain the receptacle flap to the closed mode, and a flap release mechanism. The flap release mechanism can be configured to enable the receptacle flap to be selectively attached to and separably released from the pump. The flap release mechanism can include an attachment portion of the receptacle flap and a flap attachment point fixed relative to the pump, with the attachment portion and the flap attachment point being configured to reversibly mate. In the closed mode, the receptacle flap is in a position that substantially inhibits access to the receptacle and to the syringe received by the receptacle, and in the open mode, the receptacle flap is in a position that enables access to the receptacle and to the syringe received by the receptacle.

In some cases, the flap release mechanism can be configured to be actuatable to selectively release the receptacle flap relative to the pump only when the receptacle flap is in the open mode, and not actuatable to release the receptacle flap when the receptacle flap is in the closed mode.

In some cases, the flap release mechanism can be configured to be actuatable to selectively release the receptacle flap relative to the pump without the use of any tool.

In some instances, the attachment portion of the receptacle flap can be structured with at least one subcomponent that moves between a first position and a second position. In these instances, when the receptacle flap is attached and secured to the pump, the subcomponent(s) can be deployed in the first position, and when the receptacle flap is configured to be attachable and detachable relative to the pump, the subcomponent(s) can be deployed in the second position.

In some instances, the flap attachment point can be structured with at least one subcomponent that moves between a first position and a second position. In these instances, when the receptacle flap is attached and secured to the pump, the subcomponent(s) can be deployed in the first position, and when the pump is configured for the flap to be attachable and detachable relative to the pump, the subcomponent(s) can be deployed in the second position.

In some cases, the pump can further include a clamp assembly configured to releasably secure the syringe in the receptacle. In these cases, the clamp assembly can be structured also to function as the flap latch.

In but another illustrative but non-limiting example, the disclosure provides a selectively removable flap configured to selectively attach to an infusion pump proximal a syringe receptacle of the infusion pump. The selectively removable flap can include a cover portion dimensioned to inhibit access to the syringe receptacle and to a syringe received by the receptacle when the selectively removable flap is positioned in a closed mode, and an attachment portion fixed relative to the cover portion and structured to mate with a flap attachment point of the infusion pump. The attachment portion and the flap attachment point together can provide a flap release mechanism configured to enable the selectively removable flap to be selectively attached to and separably released from the infusion pump. The flap release mechanism can be configured to enable the selectively removable flap to move between the closed mode and an open mode. In the open mode, the cover portion can be positioned to enable access to the receptacle and to the syringe received by the receptacle.

In some instances, the cover portion can be substantially non-planar and shaped to conform in part to a shape of the syringe received by the receptacle.

In some cases, the flap release mechanism can be configured to be actuatable to selectively release the receptacle flap relative to the pump only when the receptacle flap is in the open mode, and not be actuatable to selectively release the receptacle flap when the receptacle flap is in the closed mode.

In some cases, the flap release mechanism can be configured to be actuatable to selectively release the receptacle flap relative to the infusion pump without use of any tool.

In some cases, the attachment portion of the selectively removable flap can be structured with at least one subcomponent that moves between a first position and a second position. When the flap release mechanism is configured to secure the removable flap to the infusion pump, the subcomponent(s) can be deployed in the first position, and when the flap release mechanism is configured to enable the selectively removable flap to be attachable and detachable relative to the infusion pump, the subcomponent(s) can be deployed in the second position.

In some cases, the attachment portion of the selectively removable flap is structured to remain in a same configuration whether the flap release mechanism is configured to secure the selectively removable flap to the infusion pump, or is configured to enable the selectively removable flap to be attachable and detachable relative to the infusion pump.

The above summary is not intended to describe each and every example or every implementation of the disclosure. The Description that follows more particularly exemplifies various illustrative embodiments.

### BRIEF DESCRIPTION OF THE FIGURES

The following description should be read with reference to the drawings. The drawings, which are not necessarily to scale, depict several examples and are not intended to limit the scope of the disclosure. The disclosure may be more completely understood in consideration of the following description with respect to various examples in connection with the accompanying drawings, in which:

FIG. 1 is a schematic front perspective view of an example syringe pump with features to improve syringe security;

FIG. 2 is a schematic front perspective view of the syringe pump of FIG. 1 with some syringe security features shown in other functional states;

FIG. 3 is a schematic perspective view of another example of a syringe pump with features to improve syringe security;

FIG. 4 is a schematic perspective view showing details of a component detached from the pump illustrated in FIGS. 1 and 2;

FIG. 5 is a schematic rear perspective view of the pump of FIGS. 1, 2, and 4, with a back portion of a housing of the pump removed and a drive mechanism of the pump coupled to a plunger of a syringe;

FIG. 6 is a schematic rear perspective view of the syringe pump of FIGS. 1, 2, 4, and 5, with the drive mechanism of the pump retracted away from and not coupled to the plunger of the syringe;

FIG. 7 is a schematic rear perspective view of an example of internal components of a syringe pump having a clamp lock mechanism that can be linked to a state of a clutch of a drive mechanism of the pump, with the drive mechanism coupled to a plunger of a syringe;

FIG. 8 is a schematic rear perspective view of the pump of FIG. 7, with the clutch decoupled and clamp lock mechanism unlocked;

FIG. 9 is a schematic rear perspective view of an example of internal components of a syringe pump having a keyed lock operable from a front panel of the pump that is configured to actuate a clamp lock mechanism and a clutch simultaneously; and

FIG. 10 is a schematic rear partial perspective sectional view of the pump of FIG. 9 with the clamp lock mechanism unlocked and the clutch decoupled.

### DESCRIPTION

The following description should be read with reference to the drawings, in which like elements in different drawings may be numbered in like fashion. The drawings, which are not necessarily to scale, depict selected examples and are not intended to limit the scope of the disclosure. Although examples of construction, dimensions, and materials may be illustrated for the various elements, those skilled in the art will recognize that many of the examples provided have suitable alternatives that may be utilized.

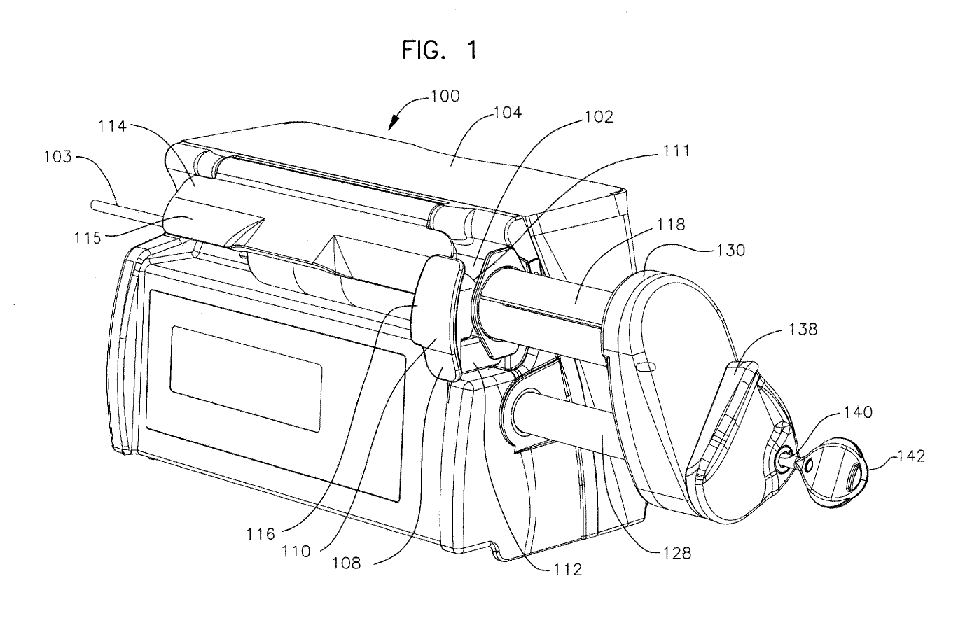

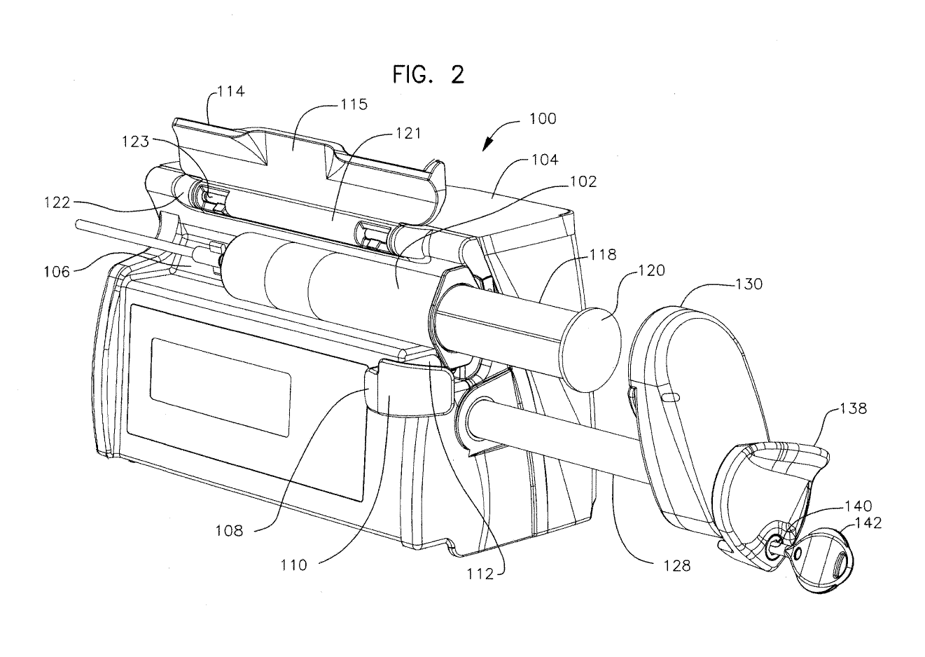

FIGS. 1 and 2 are schematic front perspective views of an example syringe pump 100 configured with features to improve syringe security. In FIG. 1, pump 100 is illustrated in what may be deemed a secure state, in which some features of pump 100 are active or otherwise positioned or deployed to provide security for syringe 102, which is illustrated as being operatively mounted to the pump. In FIG. 2, pump 100 is illustrated with some features in alternate states, relative to the configuration of FIG. 1. In the following description, some cited features are more readily viewed in one or the other of FIGS. 1 and 2, and inspection of both Figures may result in a better appreciation of the features of pump 100.

Syringe pump 100 can include a housing 104 generally enclosing an interior space and defining a space exterior to the housing. Pump 100 can include a receptacle 106 configured to receive a syringe such as syringe 102. Receptacle 106 can be formed integrally with housing 104, although this is not necessary, and the receptacle can be provided on the outside of housing 104, although such arrangement also is not necessary. Syringe 102 can be coupled to a line set 103, through which medicaments or other fluids can be delivered from the syringe reservoir.

Syringe pump 100 can include a retention mechanism configured to inhibit removal of syringe 102 from receptacle 106 when in an engaged state, and permit, allow, enable, or not inhibit removal of the syringe from the receptacle when in a disengaged state. In various configurations of pump 100, the retention mechanism can include different components or combinations of components. In some examples, the retention mechanism can include a clamp assembly 108 configured to releasably secure syringe 102 in receptacle 106. The clamp assembly 108 can include a clamp grip 110 configured to bear against the barrel of the syringe 102 as shown in FIG. 1. The portion 111 of the clamp grip 110 that is configured to bear directly against the barrel of the syringe 102 can have any suitable shape, such as a V-shape or a cupped-shape. When bearing against the syringe, the clamp grip 110 can exert a retaining force on the syringe into the receptacle 106 and toward the inside space of the housing 104. This can correspond to the engaged state of the retention mechanism. In FIG. 1, the clamp grip 110 is illustrated as bearing against the barrel of syringe 102. The clamp assembly 108 can include a clamp arm 112 connected to the clamp grip 110. The clamp arm 112 can be slidably mounted relative to the housing 104 such that the clamp grip 110 is translatable both inwardly and outwardly relative to the housing. In FIG. 2, clamp arm 112 and clamp grip 110 are illustrated as being translated outwardly relative to housing 104, such that clamp grip 110 is not bearing against the barrel of syringe 102. The clamp assembly 108 also can be structured so that the clamp grip 110 can be rotated, as illustrated in FIG. 2, although providing such rotational freedom is not required. Rotating the clamp grip 110 may be useful for positioning it away from the syringe receptacle 106 when a user wishes to remove or place a syringe in the receptacle.

If clamp arm 112 and clamp grip 110 are translated outwardly relative to housing 104, the clamp grip may not securely retain the syringe 102 in the receptacle 106. Clamp assembly 108 can include a clamp lock mechanism (not shown in FIGS. 1 and 2) configured to releasably maintain the clamp arm 112 at a syringe retention position where the clamp grip 110 exerts the retaining force to secure syringe 102 in receptacle 106. An example of a clamp lock mechanism is described in further detail elsewhere herein. When the clamp arm 112 is maintained inwardly at the syringe retention position, the retention mechanism can be considered to be constrained to the engaged state.

Syringe pump 100 can include, in some examples, a receptacle flap 114 pivotably attached to the pump. In some examples, the receptacle flap 114 is pivotably attached to the housing 104. Receptacle flap 114 can be pivotable between a closed mode and an open mode. In the closed mode, shown for example in FIG. 1, the receptacle flap 114 can be in a position that substantially inhibits or prevents access to the receptacle 106 and to the syringe 102 received by the receptacle. In the closed mode, the receptacle flap 114 (more particularly, a cover portion 115 of the receptacle flap) can substantially cover the receptacle 106. In the open mode, shown for example in FIG. 2, the cover portion 115 of the receptacle flap 114 can be in a position or range of positions that substantially permit, allow, enable, or clear access to the receptacle 106 and to the syringe 102 received by the receptacle.

When manual access to the receptacle 106 and syringe 102 is substantially inhibited by the flap 114 in the closed mode, the flap may physically obstruct or otherwise prevent touching all or part of the syringe barrel, attaching/detaching or otherwise interacting with an attachment between the syringe and infusion line set 103, removing/placing the syringe from/in the receptacle, or any other manner of access to the syringe that it may be desirable to limit. Conversely, when manual access to the receptacle 106 and syringe 102 is permitted, allowed, enabled, or cleared with the flap 114 in the open mode, syringe 102 may be manually touched, removed from receptacle 106, placed in the receptacle, attached/detached from line set 103, and the like. Although illustrated as extending substantially across receptacle 106 (along a long axis of the syringe 102), it is to be appreciated and understood that in a particular embodiment the cover portion 115 of flap 114 might extend across only a relatively smaller portion of receptacle 106, or have any suitable length or shape. Similarly, although the cover portion 115 of flap 114 as illustrated in FIG. 1 does not extend entirely around the barrel of syringe 102 (generally in the up/down direction relative to FIG. 1), it could be taller or shorter or could have any suitable shape, and could, for example, essentially obstruct a large majority of the opening of receptacle 106. Different shapes of flaps could be provided, for use with different syringe sizes and/or applications. Any suitable flap dimensions can be used. The cover portion 115 of flap 114 can be substantially non-planar and can be shaped to conform in part to a shape of the syringe 102 received by the receptacle 106. It may be appreciated that syringe pumps of the present disclosure can include receptacle flaps and/or other syringe security features that may not add appreciably to the overall dimensions or “bulk” of the pumps, as compared to competitive pumps that do not include syringe security features as disclosed herein. The syringe security features of the present disclosure may provide a significant advantage over solutions such as known lock-boxes that can significantly increase the volume occupied by a pump, relative to an easily accessible, unboxed pump.

In some cases, receptacle flap 114 can form at least part of the retention mechanism of the pump. In configurations where pump 100 includes receptacle flap 114 and clamp assembly 108, both components can work together as part of the retention mechanism. Clamp assembly 108 can be structured to constrain receptacle flap 114 to the closed mode when engaged to secure syringe 102 in receptacle 106. More specifically, in some examples clamp grip 110 of clamp assembly 108 can include structure (e.g., the left portion 116 of grip 110 in FIG. 1) that overlaps with receptacle flap 114 in the closed mode when the grip is positioned to bear against the barrel of syringe 102, thereby interfering with or otherwise inhibiting rotation of the flap to the open mode, and thus constraining the receptacle flap to the closed mode. When the clamp grip 110 is retracted outwardly away from the syringe 102, the receptacle 106, and the housing 104, as depicted in FIG. 2, grip 110 may be positioned such that it does not interfere with rotation of the flap 114 between the closed and open modes and thus does not constrain the receptacle flap to the closed mode. In some other examples, a component of the clamp assembly 108 other than the clamp grip 110 can be configured to constrain the receptacle flap 114 to the closed mode when the clamp grip is bearing against the barrel of the syringe 102. When configured to selectively constrain the receptacle flap 114 to the closed mode, the clamp assembly 108 can be considered to function as a flap latch.

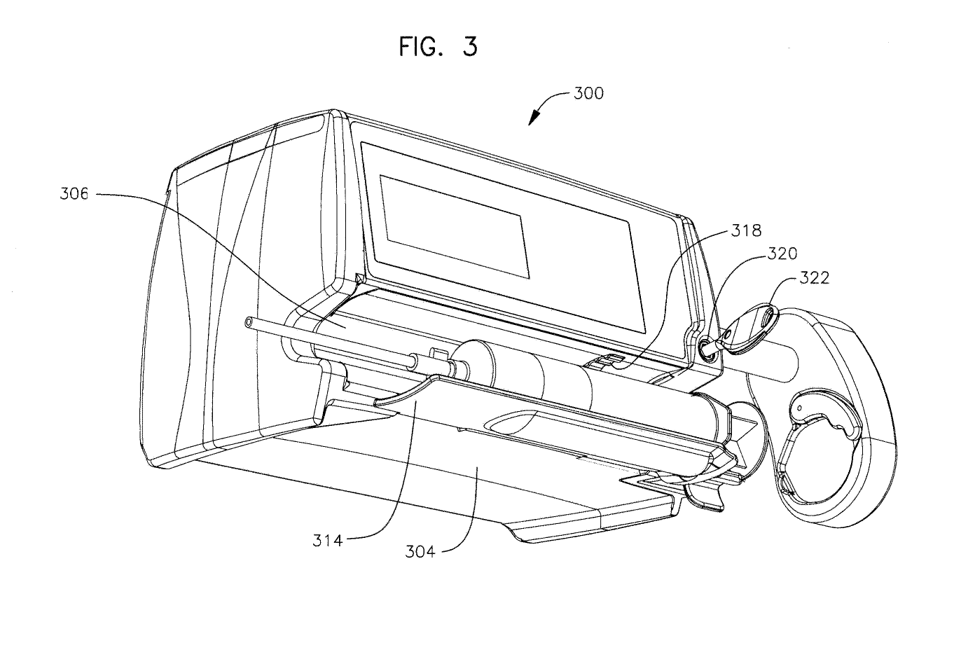

While syringe pump 100 of FIGS. 1 and 2 can be configured such that receptacle flap 114 can be constrained to the closed mode by the clamp assembly 108, it is contemplated that pumps can be configured to constrain receptacle flaps with other mechanisms. FIG. 3 is a schematic front perspective view of an infusion pump 300 having a receptacle 306 and a receptacle flap 314 pivotably coupled to a housing 304. Pump 300 can include a flap latch 318 configured to selectively constrain receptacle flap 314 in the closed mode. Flap latch 318 can include an undulating or repetitively ridged shape, as illustrated, with multiple possible interference points that can provide for constraining receptacle flap 314 at multiple possible positions along the latch to accommodate, for example, multiple possible syringe sizes, although this is not limiting and the flap latch can take any suitable shape. The receptacle flap 314 and flap latch 318 can form at least part of the retention mechanism of pump 300. Pump 300 can include a keyed lock 320 for engagement with a key 322. Keyed lock 320 can be configured to lock and unlock flap latch 318 by way of key 322, and can also be configured to lock and unlock other mechanisms of infusion pump 300, as described further elsewhere herein.

Other possible mechanisms for constraining a receptacle flap to a closed mode are contemplated. For example, a locking mechanism could be incorporated into a receptacle flap hinge (not shown). Any suitable locking mechanisms for receptacle flaps can be used.

It is to be appreciated and understood that illustrative syringe pump 300 of FIG. 3 is structured with syringe receptacle 306 provided toward a bottom portion of housing 304, whereas illustrative syringe pump 100 of FIGS. 1 and 2 is structured with syringe receptacle 106 provided toward a top portion of housing 104. While there may be relative advantages and disadvantages in some aspects with regard to top or bottom placement of syringe receptacles in various pump configurations, in general the novel and inventive features of the present disclosure may be practiced without particular regard to such placement.

It is also to be appreciated and understood that a receptacle flap need not necessarily be permanently, or even quasi-permanently, attached to its respective pump. For example, as illustrated in FIG. 2 receptacle flap 114 can be pivotably attached to syringe pump 100 via a flap release mechanism that is configured to enable the receptacle flap to be selectively attached to and separably released from the pump. Any suitable flap release mechanism can be employed.

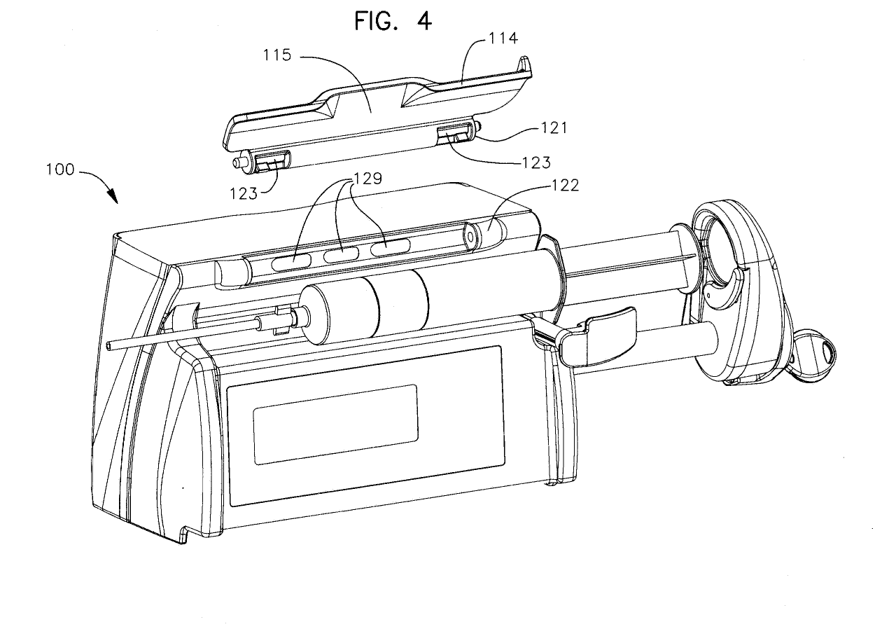

Some aspects of the flap release mechanism of pump 100 may be discerned in FIGS. 1, 2, and FIG. 4, which is a schematic front perspective view of receptacle flap 114 detached from pump 100. The flap release mechanism can include components on or of both the receptacle flap 114 and the pump 100. The receptacle flap 114 can include an attachment portion 121 fixed relative to the cover portion 115 of the flap. In some examples, all or part of the attachment portion of a receptacle flap can be formed integrally or monolithically with the cover portion of the flap (for example, in a molding process) although this is not required. The attachment portion 121 of the flap can be structured to reversibly mate with a flap attachment point 122 of the infusion pump that is fixed relative to the pump. Either or both of attachment portion 121 and flap attachment point 122 can be structured with at least one moving subcomponent. For example, receptacle flap 114 as illustrated includes left-side and right-side moving pins 123 that can be slidingly moved or deployed outwardly and inwardly between first and second positions, with the outward first position corresponding to a state where the flap release mechanism is configured to secure the flap to the infusion pump 100, and the inward second position corresponding to a state where the flap release mechanism is configured to enable the flap to be attachable and detachable relative to the pump. The pins 123 are shown in the inward second position in FIG. 2, and the outward first position in FIG. 4. Referring to FIG. 4, if the pins 123 were deployed in the inward second position (not as illustrated), the outer-most parts of the pins could be flush or nearly flush with the larger-diameter circular end faces of the attachment portion 121 of the flap 114. This arrangement, of course, is merely one example of a configuration for a flap release mechanism. In some configurations, the flap attachment point of the pump can include moving subcomponents deployable between first (flap secured) and second (flap non-secured) positions, possibly with the attachment portion of the receptacle flap essentially not having moving subcomponents. In some flap release mechanism configurations, the attachment portion of the flap can be structured to remain in a same configuration whether the flap release mechanism is configured to secure the flap to the pump, or is configured to enable the flap to be attachable and detachable relative to the pump.

Flap release mechanisms can be designed to be easily operated by a user of pump 100. In the interest of syringe security, in some configurations the flap release mechanism is actuatable to selectively attach and release the receptacle flap 114 relative to the pump 100 only when the receptacle flap is disposed in the open mode, and is not so actuatable when the receptacle flap is disposed in the closed mode. In FIG. 2, with receptacle flap 114 disposed in the open mode, features of the flap release mechanism such as moving pins 123 can be exposed to a user and can be manipulable to release or secure the flap; in FIG. 1, with receptacle flap 114 disposed in the closed mode, the same features of the release mechanism are not exposed and in effect are inaccessible to a user for manipulation. The flap release mechanism can be structured to be manipulable by an end user without the use of any tool. Alternately, in some cases, the flap release mechanism can be structured to require the use of a tool or tools for releasing receptacle flap 114 from pump 100 and/or securing the receptacle flap to the pump.

Although not illustrated in FIG. 3, it is to be understood that receptacle flap 314 and other contemplated flaps can likewise be configured similarly with release mechanisms.

Once receptacle flap 114 has been detached, it or another compatible receptacle flap may be (re-)attached to the pump 100 via the flap release mechanism. The ability to remove and/or change receptacle flap 114 easily may provide valuable adaptability to pump 100. In some embodiments, it may be desired to not have a receptacle flap at all. In other embodiments, various receptacle flaps can provide various functionality to pump 100 for various applications and/or practitioner preferences. For example, in use of syringe pumps that do not involve controlled substances, a receptacle flap might not be configured to be constrainable to a closed mode. Whereas receptacle flap 114 of syringe pump 100 of FIGS. 1 and 2 can be configured such that it can be constrained to the closed mode by the clamp assembly 108, a non-security receptacle flap could be structured differently (e.g., with a different shape) such that the clamp assembly does not interfere with or otherwise constrain the rotation of the non-security receptacle flap regardless of the position of the clamp assembly, including when the clamp grip is bearing against the barrel of the syringe. In such a configuration, the receptacle flap might not constitute part of the syringe retention system of the infusion pump.

Receptacle flap 114 can be formed from opaque, transparent, or translucent material, in any suitable color. Color can be used to color-code a syringe pump as being intended for a particular use, using colors that are by convention associated with particular therapies. For example, the color orange is often associated with enteral infusions, and the color yellow is often associated with epidural applications. It should be noted that these color associations are not universal and may vary between hospitals, institutions, regions, practices, etc. The ability to easily attach a customized receptacle flap can enable a caregiving organization to adapt the pump to the particular needs or desires of the organization. Receptacle flaps could be further customized with printed, molded, engraved, or otherwise imparted symbols, text, or any other desired marks.

Optical functionality can be provided with receptacle flaps. Transparent and/or translucent receptacle flaps can provide a window through which a syringe may be viewed, providing a user with a direct visual path to assess the current status of the syringe. All or a portion of a transparent flap can be configured as a magnifier to assist visual inspection of a syringe. In some examples, a transparent and/or translucent receptacle flap can also be used as a lens or other optic to direct or otherwise manage light used to illuminate the syringe or other aspects of the pump. For example, a receptacle flap could be used as a light guide by injecting light into an edge of the flap, and extraction features patterned into the flap could direct light out of the flap and toward the syringe. A configuration for placement of light emitters 129 of pump 100 to provide light in conjunction or cooperation with flap 114 is illustrated in FIG. 4.

Receptacle flaps such as flaps 114/314 and clamp assemblies such as assembly 108 can provide syringe security in that they can prevent or at least provide intentional physical obstacles to removal of a syringe such as syringe 102 from a syringe pump such as pumps 100 and 300. Syringe security can also be provided by maintaining control of the plunger of the syringe while the syringe is operatively mounted to a pump. Without such control, a syringe plunger could be manipulated inappropriately, which could result in delivery of a drug to a patient outside of the control of caregivers or beyond hard or soft dosage safety limits, or which could be performed to steal or otherwise obtain without permission the drug(s) contained in the syringe. In FIG. 2, the plunger 118 of syringe 102 is not controlled by pump 100 and is susceptible to manual manipulation. But in FIG. 1, the drive mechanism of pump 100 is coupled to plunger 118 of syringe 102 and can prevent inappropriate manipulation of the plunger.

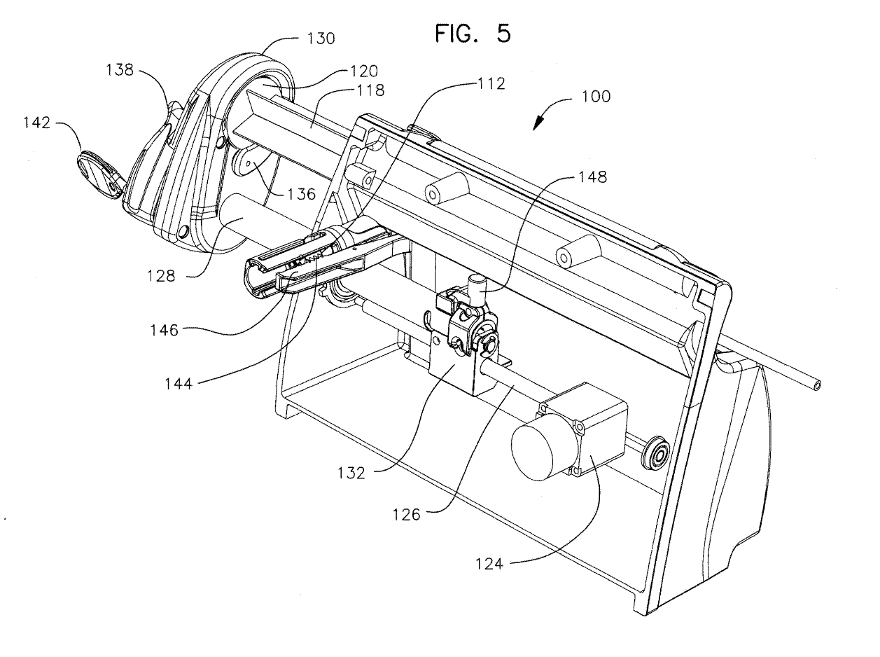

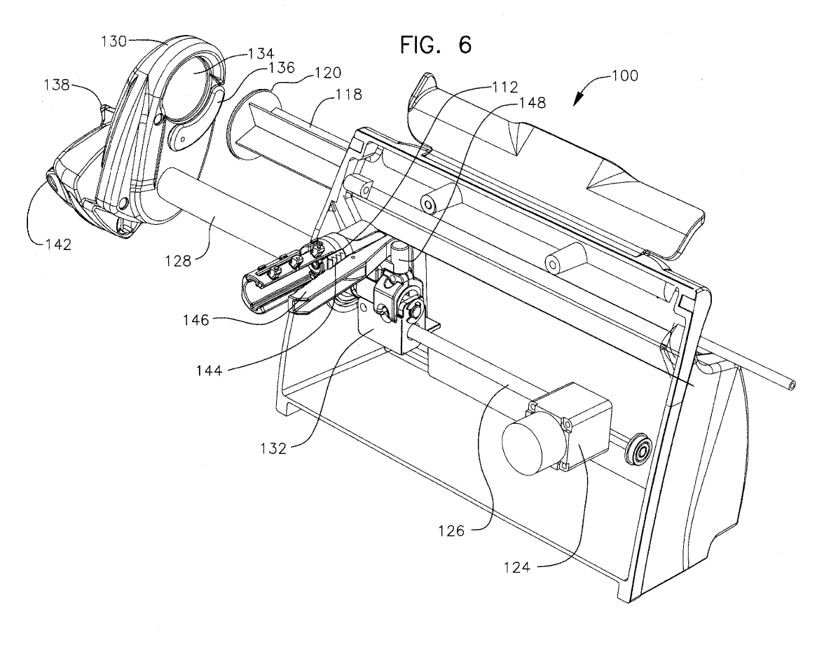

Aspects of the drive mechanism of pump 100 may be more easily viewed in FIGS. 5 and 6, which are schematic rear perspective views of syringe pump 100 of FIGS. 1 and 2, with a back portion of the housing 104 of the pump removed. In FIG. 5, the drive mechanism of pump 100 is engagingly coupled to plunger 118 of syringe 102. In FIG. 6, components of the drive mechanism of pump 100 are retracted away from syringe 102 (as well as receptacle 106 and housing 104), and the drive mechanism is not coupled to plunger 118.

In particular, in an embodiment of pump 100, the drive mechanism of the pump can include a pusher device (also commonly referred-to as a plunger driver) configured to selectively couple to thumb press 120 of plunger 118 of syringe 102, a motor 124 operatively coupleable to the pusher device, and a clutch (as further described herein) that is configured to couple the motor to the pusher device in a coupled state, and decouple the motor from the pusher device in a decoupled state. The pusher device can include multiple components that are described in further detail herein. The drive mechanism can include a lead screw 126 that is rotatably coupled to the motor 124. (Note that in the Figures, some structures such as mounts for the motor 124 and lead screw 126 have been omitted for clarity.) The clutch can selectively couple the pusher device to the lead screw 126. The clutch can incorporate any suitable clutch mechanism, and can include a halfnut, split nut, lead screw nut, etc. The pusher device of the drive mechanism can include a pusher rod 128 slidably mounted relative to receptacle 106, a pusher head 130 mounted at a first end of the pusher rod, and a clutch carriage 132 for the clutch (not visible) that is mounted at an opposite second end of the pusher rod. The clutch carriage 132, pusher rod 128, and pusher head 130 can be joined to each other essentially rigidly such that they move translationally together substantially along a longitudinal axis of the pusher rod. The clutch can be mounted to the clutch carriage 132 (in the configuration illustrated in FIGS. 4 and 5, the clutch is largely enclosed within the clutch carriage and thus obscured from view) and configured to couple the pusher device to the lead screw 126 in a coupled state and decouple the pusher device from the lead screw in a decoupled state.

When the clutch is in the coupled state, the motor 124 can be operatively coupled to the pusher device via the lead screw 126 and the clutch. The motor 124 can impart torque, and consequently, rotational motion, to lead screw 126, which the clutch can transform to linear force, and consequently, translational motion, of the pusher device. The pusher head 130 of the pusher device can be configured to exert force on thumb press 120 substantially along a longitudinal axis of the syringe 102. The pusher head 130 can include a pushing surface 134 configured to bear against the thumb press 120 of syringe 102 in an expulsion direction (to the left in FIGS. 1 & 2; to the right in FIGS. 5 & 6). Pusher head 130 can include one or more thumb press retaining clips 136 configured to retain the thumb press 120 against the pushing surface 134 when the clips are engaged, as shown in FIG. 5. The thumb press retaining clips 136 can be configured not to retain the thumb press 120 against the pushing surface 134 when the clips are disengaged, as shown in FIG. 6. Although not illustrated, it is to be understood that pusher head 130 and/or pushing surface 134 can include a device or devices such as, for example, a pressure sensor or switch that forcibly contacts the thumb press of a coupled syringe. Such a device could be used to generate an alarm signal when an occlusion occurs relative to the syringe. In addition or alternatively, such a device could provide verification that a thumb press is coupled to pusher head 130.

When the thumb press retaining clips 136 are engaged, the retention of the thumb press 120 against the pushing surface 134 of the pusher head 130 by the clips can help constrain the motion of the plunger 118 of syringe 102 only to motions corresponding to motions of the pusher head. If, in turn, the pusher device is coupled to the motor 124 via an engaged clutch, and the motor is operatively subject to a pump controller (not illustrated), then motions of the plunger 118 relative to the barrel of the syringe 102 may be constrained to motions controlled by the pump's controller. Such controlled motion of the pump's drive mechanism can help to inhibit un-commanded or otherwise inappropriate dispensing of the contents of syringe 102. For example, without such constraints, the plunger 118 could be subject to improper direct manual manipulation, or the plunger could be moved via suction in the reservoir of syringe 102. Suction can be introduced to the syringe reservoir via attached line set 103. Such suction could result from, for example, a deliberate, improper attempt to remove the contents of the syringe, or it could result accidentally.

With continued reference to FIGS. 5 and 6, the example of syringe pump 100 can include a clutch release actuator that is manually actuatable to move the clutch between coupled and decoupled states. In the example of pump 100, the clutch release actuator includes a clutch release lever 138 with pusher head 130, but this is not limiting and other implementations are contemplated. It is to be understood that the clutch release actuator can be implemented in any suitable manner. For example, in pump 100, clutch release lever 138 can be coupled to a rotatable shaft (not visible) extending inside essentially the length of pusher rod 128, with a clutch-actuating cam (not visible) coupled to the shaft at the clutch carriage 132. The shaft can be rotatable between a first position and a second position (for example via manipulation of the clutch release lever 138), with the clutch-actuating cam configured such that first and second positions of the shaft correspond to the coupled and decoupled states of the clutch, respectively.

The clutch release actuator also can be operatively coupled to a mechanism to engage and disengage thumb press retaining clips 136 so that the thumb press retaining clips are mechanically linked to the clutch such that the one or more thumb press retaining clips are engaged when the clutch is in the coupled state, and are disengaged when the clutch is in the decoupled state.

With reference again to FIGS. 1 and 2, syringe pump 100 can include a keyed lock 140 that accepts a key 142. The keyed lock can be lockable to maintain the clutch release actuator in a clutch-coupled position and unlockable to allow manipulation of the clutch release actuator between clutch-coupled and clutch-decoupled positions. In the example of pump 100, keyed lock 140 is illustrated with pusher head 130 of the pusher device, and more specifically, the keyed lock in this example is integrated with clutch release lever 138. In this configuration, the keyed lock can be configured to lock the clutch release lever 138 and also the clutch release actuator of which the clutch release lever is a component. When the clutch release lever 138 is also operatively coupled to a mechanism to engage and disengage thumb press retaining clips 136, the keyed lock 140 can effectively lock the clips in the engaged mode and the clutch in the coupled state. Other implementations of keyed locks are possible. For example, in some embodiments the clutch release lever could be omitted and the turning of a key in the lock mechanism could be directly coupled with the clutch release actuator and/or the mechanism to engage and disengage thumb press retaining clips 136. Furthermore, in some embodiments an absence of an appropriate key in the lock could restrict the clutch release actuator to the clutch coupled state and the thumb press retaining clips 136 to the engaged mode.

In the present disclosure, configurations of syringe pump 100 are described in which keyed lock 140, when locked, can maintain the clutch of the drive mechanism in the coupled state and, referring in particular to FIGS. 1 and 2, the thumb press retaining clips 136 of the pusher head in the engaged mode (if the clips are present and coupled to the clutch release actuator). The disclosure also describes retention mechanisms that can include clamp assemblies 108 and/or receptacle flaps 114/314 as aforementioned, that can act individually or cooperatively to retain syringes in syringe receptacles of the pumps. The present disclosure contemplates further interconnections between syringe security functions that can simplify caregiver workloads.

Regarding the clamp assembly 108 of the retention mechanism as shown in FIGS. 1 and 2, FIGS. 5 and 6 provide views of one example of a clamp lock mechanism (within housing 104) corresponding to clamp assembly 108 that can include a ratchet 144 coupled to clamp arm 112, and a pivotably-mounted pawl 146 secured relative to the pump 100, and more specifically, secured relative to the syringe receptacle 106 and/or housing 104. In some examples of the present disclosure, in order to simplify caregiver workloads, the clamp lock mechanism can be structured and configured in cooperation with the drive mechanism such that the clamp lock mechanism can be released/unlocked depending upon the state of the drive mechanism. In various examples, release of the clamp lock mechanism can be linked to different states of the drive mechanism.

For example, pump 100 can be configured such that the clamp lock mechanism is released when the pusher device is retracted essentially to a fully-retracted position. For such a configuration, the pusher device can include a clamp lock release structure 148 (also referred to as a “retention releaser”) secured relative to the pusher rod 128. The clamp lock release structure 148 can be provided or included with the clutch carriage 132, with the clamp lock release structure secured relative to the clutch carriage and/or formed integrally with the clutch carriage. When the pusher head 130 is retracted away from housing 104, receptacle 106, and thumb press 120 of syringe 102 (if present), clamp lock release structure 148 can be drawn toward pawl 146. When the pusher device is retracted essentially to a fully-retracted position, clamp lock release structure 148 can be drawn into contact with pawl 146, pivoting the pawl such that it releases ratchet 144 thereby releasing clamp arm 112. In this way, release of the retention mechanism, of which clamp assembly 108 is a component, can be tied to the position of the pusher device of the drive mechanism. Stated another way, in this arrangement, as the pusher head 130 is retracted away from the receptacle 106, a retention releaser (the clamp lock release structure 148) is brought into proximity of the retention mechanism (the ratchet 144 and pawl 146 are components of the retention mechanism) and releases the retention mechanism from the engaged state. Other hardware implementations to achieve the same effect are contemplated in the present disclosure. In an example, the retention releaser could be a magnet that, when brought into proximity of a magnetically-actuatable retention mechanism, provides a magnetic field sufficient to release the retention mechanism. Additional mechanisms to link release of a retention mechanism to position of a drive mechanism are possible, and the examples described or otherwise contemplated herein may be provided alone or in various combinations with each other provided that they function in accordance with subject matter hereof.

In the arrangement of pump 100 as described, the clamp assembly 108 of the retention mechanism can be released when the pusher device is retracted essentially to a fully-retracted position. Such a retraction of the pusher device could, for example, be driven by motor 124 with the clutch coupled, although generally the controller of pump 100 would not be programmed to command such a motion of the pusher device. In some examples, the controller of pump 100 can be programmed specifically to prevent motor 124 from retracting the pusher device to a fully-retracted position. Typically such a retraction would be executed by manual manipulation of the pusher device with the clutch decoupled.

To release and remove syringe 102 from pump 100, an authorized user can, for example, perform the following sequence: Unlock the keyed lock 140 with a corresponding key 142 if the pump is locked. With the keyed lock 140 unlocked, rotate the clutch release lever 138 from the coupled position (as illustrated in FIGS. 1 and 5) to the decoupled position (FIGS. 2 and 6). This rotation of the clutch release lever 138 can decouple the clutch via the clutch release actuator and can simultaneously retract the thumb press retaining clips 136. With the clutch decoupled, grasp the pusher head 130 and fully retract the pusher device (away or outwardly from housing 104, receptacle 106, and thumb press 120). When the pusher head 130 is fully retracted, the retention mechanism is released by the retention releaser—that is, the clamp assembly 108 is unlocked, allowing the clamp grip 110 to be retracted from syringe 102, and receptacle flap 114 (if present) can then be manipulated to the open mode, allowing removal of the syringe from the receptacle 106. If necessary or desired, this sequence of manipulation can be performed with one hand.

In accordance with the aforedescribed example of a sequence for release and removal of a syringe 102 from pump 100, it is to be understood that the keyed lock 140, retention mechanism, and drive mechanism can be configured to operate in a coordinated manner such that, if: (a) the keyed lock is locked; (b) the retention mechanism is in the engaged state; and (c) the pusher is coupled to the thumb press of the syringe (which implies that the pusher is not fully retracted), then as long as the keyed lock remains locked (that is, for an interval of time commencing when all three conditions (a), (b), and (c) are met and persisting until the keyed lock is first unlocked after the commencement of the time interval), the retention mechanism is constrained to the engaged state and the clutch is constrained to the coupled state. When so configured, if it is desired to remove the syringe 102 from the receptacle 106, the keyed lock 140 would need to be unlocked to permit or allow decoupling of the clutch, and the clutch would need to be decoupled to retract the pusher mechanism and release the retention mechanism when fully retracted.

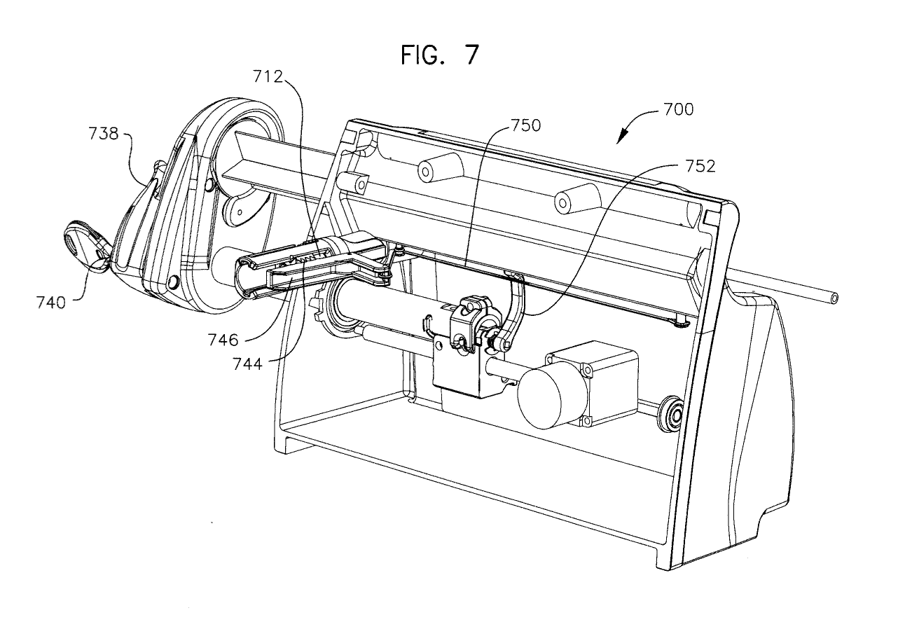

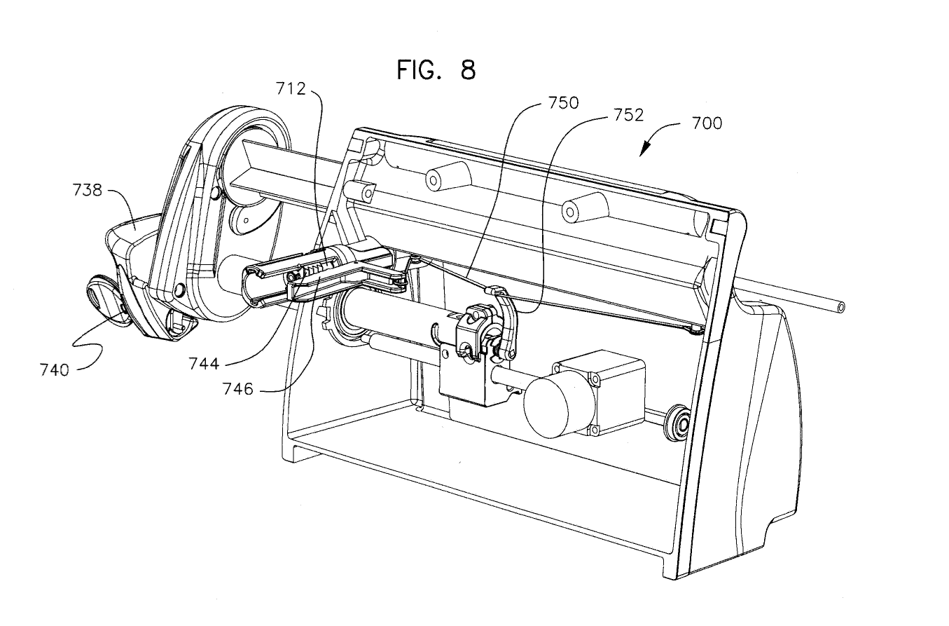

FIGS. 7 and 8 are schematic perspective views of a syringe pump 700 similar in many aspects to syringe pump 100, but having a clamp lock mechanism that can be linked to a state of its drive mechanism differently from how the clamp lock mechanism and drive mechanism of pump 100 are linked (in which the clamp lock mechanism can be linked to the position of the drive mechanism). In syringe pump 700, the clamp lock mechanism can be linked to the state of the clutch of the drive mechanism. FIGS. 7 and 8 are rendered generally from the back side of the pump 700, with a back portion of the housing of the pump removed.

Pump 700 can include a linkage mechanism mechanically coupling the clutch release actuator with the retention mechanism. The retention mechanism of pump 700 can include a clamp lock mechanism that is in some aspects similar to, but in other aspects different from, that of pump 100, with a ratchet 744 coupled to clamp arm 712, and a pivotably-mounted pawl 746 secured relative to the pump 700. Pawl 746 of the clamp lock mechanism can be operatively coupled to a release cable 750 as part of the linkage mechanism. Pawl 746 and release cable 750 can be configured such that when the end of the cable coupled to the pawl is displaced to a release point, the pawl pivots such that it releases ratchet 744, thereby releasing clamp arm 712 of the retention mechanism (see, for example, FIG. 8). To selectively control displacement of the release cable 750, the linkage mechanism can also include a tensioning arm 752 rotatably coupled to a rotatable shaft of a clutch release actuator extending inside pusher rod 728 (not visible; similarly configured in pump 700 as in pump 100), the tensioning arm generally extending from the rotatable shaft radially. The shaft can be rotatable between a first position and a second position (for example via manipulation of clutch release lever 738), with a coupled clutch-actuating cam configured such that the first and second positions of the shaft correspond to the coupled and decoupled states of the clutch, respectively. The tensioning arm 752 can be slidably or otherwise translationally coupled to the release cable 750 and configured to displace the release cable correspondingly as it rotates in accordance with the rotatable shaft, and in particular can be configured such that when the shaft is in the first position (illustrated in FIG. 7) the tensioning arm 752 does not displace the release cable 750 sufficiently to release ratchet 744 (i.e., the clamp lock mechanism remains locked), but when the shaft is in the second position (illustrated in FIG. 8) the tensioning arm does displace the release cable at least to a release point, sufficient to pivot the pawl 746 and release the ratchet. Thus as described, the linkage mechanism can be configured to mechanically link the clutch release actuator with the clamp lock mechanism such that when the clutch release actuator is in the clutch-decoupled position, the clamp lock mechanism is released. As the clutch is moved between the coupled and decoupled states, the clamp lock mechanism can correspondingly be moved between locked and released states. Stated another way, the linkage mechanism can be configured to mechanically couple the clutch release actuator with the retention mechanism such that when the clutch release actuator is in the clutch-decoupled position, the retention mechanism is not constrained to the engaged state.

Pump 700 can include a keyed lock (not visible) that can be configured, for example, to lock various aspects of syringe security features of the pump. Keyed lock 740 can be configured, for example, to prevent manipulation of clutch release lever 738 when locked, consequently locking the clutch to the coupled state. Through the linkage mechanism, keyed lock 740 can also serve to lock the clamp assembly to a syringe retention position.

To release and remove a syringe from pump 700, an authorized user can, for example, perform the following sequence: Unlock the keyed lock 740 with a corresponding key if the pump is locked. With the keyed lock unlocked, rotate the clutch release lever 738 from the coupled position (as illustrated in FIG. 7) to the decoupled position (FIG. 8). This rotation of the clutch release lever 738 can simultaneously decouple the clutch via the clutch release actuator, retract the thumb press retaining clips 736, and release the clamp lock mechanism, allowing the clamp to be retracted from the syringe, and the receptacle flap (if present) can then be flipped to the open mode, allowing removal of the syringe from the receptacle. If necessary or desired, this sequence of manipulation can be performed with one hand.

In accordance with the aforedescribed example of a sequence for release and removal of a syringe from pump 700, it is to be understood that the keyed lock 740, retention mechanism, and drive mechanism can be configured to operate in a coordinated manner (via, for example, the linkage mechanism) such that, if: (a) the keyed lock is locked; and (b) the retention mechanism is in the engaged state, then as long as the keyed lock remains locked, the clutch is constrained to the coupled state and the retention mechanism is constrained to the engaged state. When so configured, if it is desired to remove the syringe from the receptacle, the keyed lock 740 would need to be unlocked to permit or allow decoupling the clutch and releasing the retention mechanism.

In some embodiments, syringe pumps can be configured similarly to pump 700 of FIGS. 7 and 8, but a clutch release lever could be omitted. In such an embodiment without a clutch release lever, the turning or other physical actuation of a key in the lock mechanism could be directly coupled with the clutch release actuator to decouple the clutch and release the retention mechanism from the engaged state. In some embodiments, the mere absence of an appropriate key in the lock could restrict the clutch release actuator to the clutch coupled state and thereby inhibit or prevent release of the retention mechanism from the engaged mode.

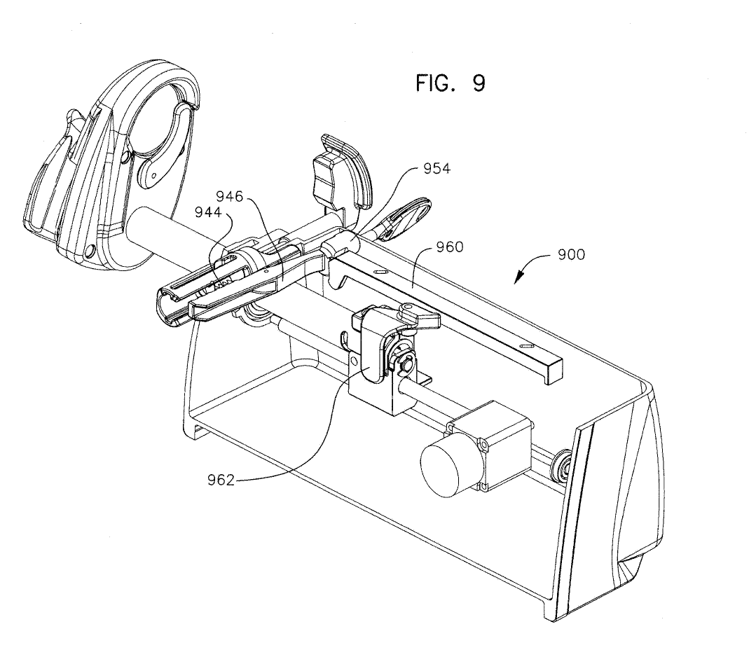

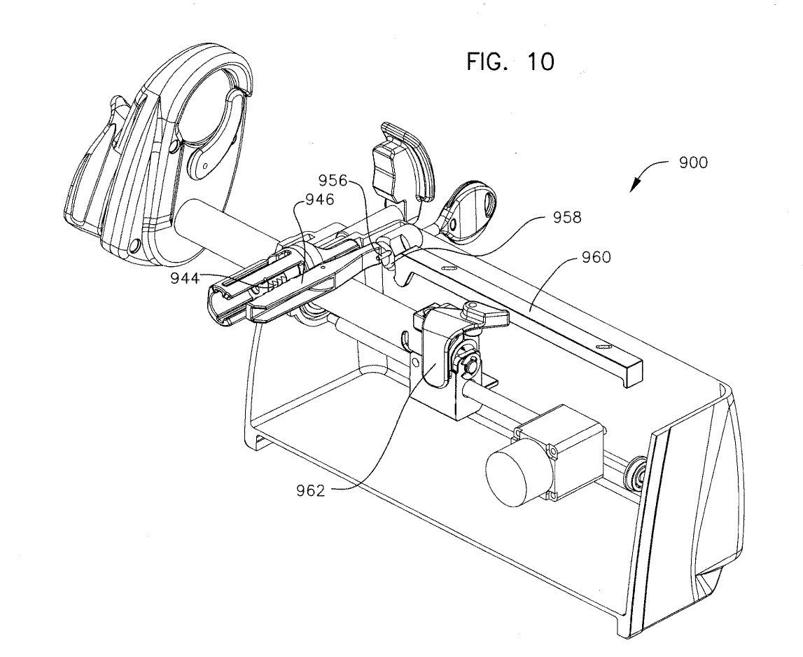

Other configurations of linkages between retention mechanisms and drive mechanisms in syringe pumps are contemplated in the present disclosure. For example, FIGS. 9 and 10 depict schematic perspective partial sectional views of a syringe pump 900 similar in several aspects to syringe pumps 100 and 700, but with a keyed lock 954 operable from the front of the pump that is configured to actuate the clamp lock mechanism and the clutch simultaneously. FIGS. 9 and 10 are rendered generally from the back side of the pump 900, with a back portion of the housing of the pump removed. For further clarity, pump structures that are above a horizontal plane sectional cut also are not shown.

The barrel of keyed lock 954 can include or be coupled to structures configured to link rotation of the barrel with actuation of the clamp lock mechanism and the clutch. In FIG. 9, the keyed lock 954 is depicted in the locked position, with the clamp lock mechanism locking the clamp assembly and the clutch so coupled. In FIG. 10, the keyed lock 954 is depicted as having been rotated to an unlocked position. With rotation of the lock barrel from the locked to unlocked positions, a barrel pin 956 can move and bear against pivotably-mounted pawl 946 such that the pawl releases ratchet 944 of the clamp lock mechanism. Simultaneously, a barrel cam 958 can rotate and thereby move slide bar 960. Motion of slide bar 960 can be transferred via spring-loaded clutch linker 962 to decouple the clutch when the keyed lock 954 is rotated to an unlocked position. This is just one example of how motion of a keyed lock could be coupled to a retention mechanism and a clutch such that rotation of the keyed lock simultaneously actuates both devices. It is to be appreciated, however, that any suitable mechanical arrangements and components in accordance with subject matter hereof could be used to provide the actions and functions as described or otherwise contemplated herein.

In other embodiments of syringe pumps (not illustrated) according to subject matter hereof, a pump can include a keyed lock that is lockable to maintain a retention mechanism in an engaged state, and unlockable to release the retention mechanism from the engaged state, but that is not configured to decouple a clutch directly. For changing the state of the clutch, the pump could include a linkage mechanism mechanically coupling the retention mechanism with the clutch such that when the retention mechanism is in the engaged state, the clutch is constrained to the coupled state, and when the retention mechanism is in the disengaged state, the clutch is not constrained to the coupled state. With a pump having such a linkage mechanism, manipulation of the retention assembly after its unlocking via the keyed lock could decouple the clutch. For example, a keyed lock could unlock a clamp lock mechanism (but not release the clutch) in a similar manner as in the example of pump 900 of FIGS. 9 and 10. After the clamp assembly is unlocked, a user could retract (or pull) the clamp grip away from the syringe. Motion of the clamp arm (from the retraction) could be transferred to a rotational motion of a shaft of a clutch actuation mechanism by any suitable means such as, for example, via a rack and pinion arrangement, or via a belt linkage. A cam rotating with the shaft could then decouple the clutch. The foregoing description is just one example of a particular mechanical configuration to link motion of a retention mechanism to a clutch release. It is to be appreciated, however, that any suitable mechanical arrangements and components in accordance with subject matter hereof could be used to provide the actions and functions as described or otherwise contemplated herein.

Many infusion pumps of the present disclosure can include a “keyed lock.” A keyed lock can be an entirely manually-actuated mechanical mechanism, with a mechanical key that is manipulable by a user to actuate the lock mechanism without any external power source, such as electrical power. Some embodiments of the present disclosure provide arrangements and/or mechanisms linking a keyed lock to some or all of retention mechanisms, syringe clamps, receptacle flaps, pushers, clutches, thumb press retaining clips, etc., such that the states of the various components can have interconnections and interdependencies (for example, as described herein, the clamp lock mechanism of pump 100 can be released when the pusher device is essentially fully retracted). All of the described interdependencies can be implemented in entirely mechanical ways that do not require electrical power. Mechanical power to realize and actuate the arrangements and/or mechanisms can be supplied by a user—for example, by manipulating a lock mechanism, a clutch release lever, a pusher device, and so on. In syringe pumps that do not require external artificially-provided power to actuate or de-actuate syringe security mechanisms, caregivers can access the syringe as desired or required regardless of the presence (or lack thereof) of electrical power, thus providing syringe security features without compromising access to the syringe when electrical power is lost or otherwise absent.

While the present disclosure contemplates syringe pumps featuring entirely mechanical keyed locks and entirely mechanically-implemented interdependences between syringe security features, as summarized in the preceding paragraph, it is not limited to such entirely mechanical syringe security systems. For example, in some embodiments a “keyed lock” can feature a “key” bearing information in a manner that is not entirely (or at all) mechanical, such as a swiped or tapped card, a code entered by keypad or transmitted electronically, biometric information, an radio-frequency identification (RFID) or near field communication (NFC) device, or any other suitable key that may or may not include a physical key. In some embodiments, a syringe pump can include a keyed lock that is not entirely mechanical in conjunction with entirely mechanical interdependencies or interlink mechanisms between other syringe security features. Such a pump could feature a mechanical override of the keyed lock, such that the mechanically interlinked syringe security features could still be operated, for example, in case of power failure. In other examples, syringe security features could be provided and interlinked with more extensive reliance on electrical/electronic devices, such as electric motors, solenoids, and electronically-implemented logic to realize interlinks and interdependencies between various syringe security features. For example, presentation of an authorized electronic key could result in electrical power being supplied to solenoids and/or electrical motors that could release a clamp lock and decouple a clutch.

This disclosure is to be understood to be not limited to the particular examples described herein, but rather should be understood to cover all aspects of the disclosure and equivalents thereof. Various modifications, processes, and components, as well as numerous structures to which the disclosure can be applicable, will be readily apparent to those of skill in the art upon review of the instant specification.

1. An infusion pump, comprising:
a receptacle configured to receive a syringe;
a retention mechanism configured to inhibit removal of the syringe from the receptacle when in an engaged state, and to enable removal of the syringe from the receptacle when in a disengaged state;
a drive mechanism, including:
a pusher configured to selectively couple to a thumb press of the syringe;
a motor operatively coupleable to the pusher;
a clutch configured to couple the motor to the pusher in a coupled state, and decouple the motor from the pusher in a decoupled state;
a keyed lock configured to operate in coordination with the retention mechanism and the drive mechanism such that, if:
(a) the keyed lock is locked;
(b) the retention mechanism is in the engaged state; and
(c) the pusher is coupled to the thumb press of the syringe,
then as long as the keyed lock remains locked, the retention mechanism is constrained to the engaged state and the clutch is constrained to the coupled state.

2. The pump of claim 1, wherein the pusher includes a pusher head, the pusher head having a pushing surface configured to bear against the thumb press in an expulsion direction, and the pusher head further having at least one thumb press retaining clip configured to retain the thumb press against the pushing surface when the at least one thumb press retaining clip is engaged, and not retain the thumb press against the pushing surface when the at least one thumb press retaining clip is disengaged.

3. The pump of claim 1, wherein the at least one thumb press retaining clip is mechanically linked to the clutch such that the at least one thumb press retaining clip is engaged when the clutch is in the coupled state, and is disengaged when the clutch is in the decoupled state.

4. The pump of claim 1, wherein the keyed lock is disposed on the pusher.

5. The pump of claim 4, further comprising a clutch release actuator actuatable to move the clutch between the coupled and decoupled states, wherein the keyed lock is lockable to maintain the clutch release actuator in a clutch-coupled position and unlockable to enable manipulation of the clutch release actuator between clutch-coupled and clutch-uncoupled positions.

6. The pump of claim 5, further comprising a linkage mechanism mechanically coupling the clutch release actuator with the retention mechanism, wherein when the clutch release actuator is in a clutch-decoupled position, the retention mechanism is not constrained to the engaged state.

7. The pump of claim 5,
wherein the pusher includes:
a pusher rod slidably mounted relative to the receptacle;
a pusher head mounted at a first end of the pusher rod, the pusher head configured to exert force on the thumb press in an expulsion direction; and
a retention releaser secured relative to the pusher rod; and
wherein the pusher, when the clutch is in the decoupled state, can be retracted such that the pusher head is retracted away from the receptacle and the retention releaser is drawn toward the retention mechanism; and further, when the pusher is retracted essentially to a fully-retracted position, the retention releaser is brought into proximity of the retention mechanism and releases the retention mechanism from the engaged state.

8. The pump of claim 1, wherein the keyed lock is lockable to maintain the retention mechanism in the engaged state, and unlockable to enable release of the retention mechanism from the engaged state.

9. The pump of claim 8, further comprising a linkage mechanism mechanically coupling the retention mechanism with the clutch such that when the retention mechanism is in the engaged state, the clutch is constrained to the coupled state, and when the retention mechanism is in the disengaged state, the clutch is not constrained to the coupled state.

10. The pump of claim 1, wherein the retention mechanism includes:
a clamp assembly configured to releasably secure the syringe in the receptacle, including:
a clamp grip configured to bear against a barrel of the syringe, and when so bearing, exert a retaining force on the syringe into the receptacle;
a clamp arm connected to the clamp grip, the clamp arm slidably mounted relative to the receptacle such that the clamp grip is translatable inward and outward relative to the receptacle; and
a clamp lock mechanism configured to releasably maintain the clamp arm at a syringe retention position where the clamp grip exerts the retaining force on the syringe;

11. The pump of claim 10, further comprising a receptacle flap pivotably attached to the pump, the flap pivotable between a closed mode and an open mode;
wherein in the closed mode, the receptacle flap is in a position that substantially inhibits access to the receptacle and to the syringe received by the receptacle; and
wherein in the open mode, the receptacle flap is in a position that substantially clears access to the receptacle and to the syringe received by the receptacle.

12. The pump of claim 11, wherein the receptacle flap and clamp assembly are structured and configured such that the clamp assembly, when the clamp grip is bearing against the barrel of the syringe, constrains the receptacle flap to the closed mode.

13. The pump of claim 11, wherein the receptacle flap and clamp assembly are structured and configured such that the clamp assembly, when the clamp grip is bearing against the barrel of the syringe, does not constrain the receptacle flap to the closed mode.

14. The pump of claim 1, further comprising a receptacle flap pivotably attached to the pump, the flap pivotable between a closed mode and an open mode;
wherein in the closed mode, the receptacle flap is in a position that generally inhibits access to the receptacle and to the syringe received by the receptacle; and
wherein in the open mode, the receptacle flap is in a position that generally substantially clears access to the receptacle and to the syringe received by the receptacle.

15. The pump of claim 14, further comprising a flap latch configured to selectively constrain the receptacle flap to the closed mode, such that the receptacle flap and flap latch form at least part of the retention mechanism.

16. The pump of claim 14, wherein the receptacle flap is pivotably attached to the pump via a flap release mechanism configured to enable the receptacle flap to be selectively attached to and separably released from the pump.

17. The pump of claim 16, wherein the flap release mechanism is actuatable to selectively release the receptacle flap relative to the pump only when the receptacle flap is in the open mode, and is not actuatable to release the receptacle flap when the receptacle flap is in the closed mode.

18. An infusion pump, comprising:
a receptacle configured to receive a syringe;
a clamp assembly configured to releasably secure the syringe in the receptacle, including:
a clamp grip configured to bear against a barrel of the syringe, and when so bearing, exert a retaining force on the syringe into the receptacle;
a clamp arm connected to the clamp grip, the clamp arm slidably mounted relative to the receptacle such that the clamp grip is translatable inward and outward relative to the receptacle; and
a clamp lock mechanism configured to releasably maintain the clamp arm at a syringe retention position where the clamp grip exerts the retaining force on the syringe; and
a drive mechanism, including:
a pusher device, including:
a pusher rod slidably mounted relative to the receptacle;
a pusher head mounted at a first end of the pusher rod, the pusher head configured to exert force on a thumb press of the syringe; and
a clutch carriage mounted at a second end of the pusher rod, the clutch carriage including a clamp lock release structure;
a lead screw;
a motor coupled to the lead screw; and
a clutch mounted to the clutch carriage and configured to couple the pusher device to the lead screw in a coupled state and decouple the pusher device from the lead screw in a decoupled state;
wherein the pusher device, when the clutch is in the decoupled state, can be retracted such that the pusher head is drawn away from the receptacle and the clutch carriage is drawn toward the clamp lock mechanism; and further, when the pusher device is retracted essentially to a fully-retracted position, the clamp lock release structure is brought into proximity of the clamp lock mechanism and actuates the clamp lock mechanism to release the clamp arm.

19. The pump of claim 18, further comprising a keyed lock, wherein the clutch is lockable to the coupled state by the keyed lock.

20. The pump of claim 19, wherein the keyed lock is with the pusher head.

21. The pump of claim 19, further comprising an actuatable clutch release actuator, the clutch release actuator lockable to a clutch-coupled position by the keyed lock.

22. The pump of claim 18, wherein the clamp lock mechanism includes a ratchet.

23. An infusion pump, comprising:
a receptacle configured to receive a syringe;
a clamp assembly configured to releasably secure the syringe in the receptacle, including:
a clamp grip configured to bear against a barrel of the syringe, and when so bearing, exert a retaining force on the syringe into the receptacle;
a clamp arm connected to the clamp grip, the clamp arm slidably mounted relative to the receptacle such that the clamp grip is translatable inward and outward relative to the receptacle; and
a clamp lock mechanism configured to releasably maintain the clamp arm at a syringe retention position where the clamp grip exerts the retaining force on the syringe;
a drive mechanism, including:
a pusher configured to selectively couple to a thumb press of the syringe;
a motor operatively coupleable to the pusher; and
a clutch configured to couple the motor to the pusher in a coupled state, and decouple the motor from the pusher in a decoupled state;
a linkage mechanism configured to couple the clutch and the clamp lock mechanism such that as the clutch is moved between the coupled and decoupled states, the clamp lock mechanism is correspondingly moved between locked and released states; and
a keyed lock, with the clutch, clamp lock mechanism, and linkage mechanism configured such that the clutch and clamp lock mechanism are lockable via the keyed lock to the coupled state and locked state, respectively.

24. The pump of claim 23, wherein the linkage mechanism includes:
a shaft rotatable between a first position and a second position;
a clutch-actuating cam coupled to the shaft and configured such that the first and second positions of the shaft correspond to the coupled and decoupled states of the clutch;
a release cable coupled to the clamp lock mechanism configured to release the clamp arm when an end of the cable coupled to the clamp lock mechanism is displaced to a release point;
an arm, rotatably coupled to the shaft and coupled to the release cable such that the arm displaces the clamp release cable correspondingly with rotations of the shaft, the second position of the shaft corresponding to displacement of the cable at least to the release point, the first position of the shaft corresponding to displacement of the cable insufficient to release the clamp arm.

25. The pump of claim 23, wherein the keyed lock is disposed on the pusher.

26. An infusion pump, comprising:
a receptacle configured to receive a syringe;
a retention mechanism configured to inhibit removal of the syringe from the receptacle when in an engaged state, and to enable removal of the syringe from the receptacle when in a disengaged state;
a drive mechanism, including:
a pusher configured to selectively couple to a thumb press of the syringe;
a motor operatively coupleable to the pusher;
a clutch configured to couple the motor to the pusher in a coupled state, and decouple the motor from the pusher in a decoupled state; and
a keyed lock configured to operate in coordination with the retention mechanism and the drive mechanism such that, if:
(a) the keyed lock is locked; and
(b) the retention mechanism is in the engaged state;
then as long as the keyed lock remains locked, the retention mechanism is constrained to the engaged state and the clutch is constrained to the coupled state.

27. An infusion pump, comprising:
a receptacle configured to receive a syringe;
a receptacle flap pivotably attached to the pump, the flap pivotable between a closed mode and an open mode;
a flap latch configured to selectively constrain the receptacle flap to the closed mode; and
a flap release mechanism configured to enable the receptacle flap to be selectively attached to and separably released from the pump, the flap release mechanism including an attachment portion of the receptacle flap and a flap attachment point fixed relative to the pump, the attachment portion and the flap attachment point being configured to reversibly mate,
wherein in the closed mode, the receptacle flap is in a position that substantially inhibits access to the receptacle and to the syringe received by the receptacle; and
wherein in the open mode, the receptacle flap is in a position that enables access to the receptacle and to the syringe received by the receptacle.

28. The pump of claim 27, wherein the flap release mechanism is configured to be actuatable to selectively release the receptacle flap relative to the pump only when the receptacle flap is in the open mode, and is not actuatable to release the receptacle flap when the receptacle flap is in the closed mode.

29. The pump of claim 27, wherein the flap release mechanism is configured to be actuatable to selectively release the receptacle flap relative to the pump without use of any tool.

30. The pump of claim 27, wherein the attachment portion of the receptacle flap is structured with at least one subcomponent that moves between a first position and a second position, wherein when the receptacle flap is attached and secured to the pump, the at least one subcomponent is deployed in the first position, and when the receptacle flap is configured to be attachable and detachable relative to the pump, the at least one subcomponent is deployed in the second position.

31. The pump of claim 27, wherein the flap attachment point is structured with at least one subcomponent that moves between a first position and a second position, wherein when the receptacle flap is attached and secured to the pump, the at least one subcomponent is deployed in the first position, and when the pump is configured for the flap to be attachable and detachable relative to the pump, the at least one subcomponent is deployed in the second position.

32. The pump of claim 27, further comprising a clamp assembly configured to releasably secure the syringe in the receptacle, wherein the clamp assembly is structured also to function as the flap latch.

33. A selectively removable flap configured to selectively attach to an infusion pump proximal a syringe receptacle of the infusion pump, comprising:
a cover portion dimensioned to inhibit access to the syringe receptacle and to a syringe received by the receptacle when the selectively removable flap is positioned in a closed mode; and
an attachment portion fixed relative to the cover portion and structured to mate with a flap attachment point of the infusion pump,
wherein the attachment portion and the flap attachment point together provide a flap release mechanism configured to enable the selectively removable flap to be selectively attached to and separably released from the infusion pump;
further wherein the flap release mechanism is configured to enable the selectively removable flap to move between the closed mode and an open mode, wherein in the open mode, the cover portion is positioned to enable access to the receptacle and to the syringe received by the receptacle.

34. The flap of claim 33, wherein the cover portion is substantially non-planar and shaped to conform in part to a shape of the syringe received by the receptacle.

35. The flap of claim 33, wherein the flap release mechanism is configured to be actuatable to selectively release the receptacle flap relative to the pump only when the receptacle flap is in the open mode, and not to be actuatable to selectively release the receptacle flap when the receptacle flap is in the closed mode.

36. The flap of claim 33, wherein the flap release mechanism is configured to be actuatable to selectively release the receptacle flap relative to the infusion pump without use of any tool.

37. The flap of claim 33, wherein the attachment portion of the selectively removable flap is structured with at least one subcomponent that moves between a first position and a second position, wherein when the flap release mechanism is configured to secure the removable flap to the infusion pump, the at least one subcomponent is deployed in the first position, and when the flap release mechanism is configured to enable the selectively removable flap to be attachable and detachable relative to the infusion pump, the at least one subcomponent is deployed in the second position.

38. The flap of claim 33, wherein the attachment portion of the selectively removable flap is structured to remain in a same configuration whether the flap release mechanism is configured to secure the selectively removable flap to the infusion pump, or is configured to enable the selectively removable flap to be attachable and detachable relative to the infusion pump.
